(19) United States
(12) Patent Application Publication
(10) Pub. No.: US 2012/0308477 A1
(43) Pub. Date: Dec. 6, 2012

(54) BRIEF RADIOIMMUNOTHERAPY

(75) Inventors: Vincent BOUDOUSQ, Clapiers (FR); Isabelle Navarro-Teulon, Saint Gely Du Fesc (FR); Jean-Pierre Pouget, Notre Dame De Londres (FR); Andre Pelegrin, Montpellier (FR)

(73) Assignees: CENTRE REGIONAL DE LUTTE CONTRE LE CANCER DE MONTPELLIER; CENTRE HOSPITALIER UNIVERSITAIRE NIMES

(21) Appl. No.: 13/068,848

(22) Filed: May 30, 2011

(51) Int. Cl. A61K 51/10; A61N 5/00; A61P 35/00

(52) U.S. Cl. 424/1.49; 600/3

(57) ABSTRACT

The present invention relates to a method of treating a subject having a cancer in a body cavity characterized in that it comprises the steps of:
administering in said body cavity of the subject radiolabeled binding molecules which bind to an antigen expressed by cancerous cells;
washing the body cavity to remove unbound radiolabeled molecules.

The present invention also relates to a body cavity perfusing system for carrying out said method.

### FIELD OF THE INVENTION

The invention concerns radioimmunotherapy.

More precisely, the invention relates to a method of treating a subject having a cancer in a body cavity.

### BACKGROUND OF THE INVENTION

Radioimmunotherapy (RIT) has been recently used as a new cancer therapy. Toxicity of RIT is generally less detrimental for healthy tissues than chemotherapy.

RIT consists in using a radiolabeled antibody, i.e. labeled with a source of ionizing radiations, to deliver a lethal dose to a target cell, in particular a tumor cell. This is made possible by choosing an antibody with specificity for a tumor-associated antigen.

The ability for the antibody to specifically bind to a tumor-associated antigen increases the dose delivered to the tumor cells while decreasing the dose to normal tissues. Radionuclides Bi-213, Ac-225 or Y-90 are for example used because of their strong activity and their half-life of several hours for one of the most promising areas of RIT: the treatment of non-Hodgkin's lymphoma.

But lymphoma only represent a minority of cancers. Common lung, breast, prostate, colon or pancreas cancers are nearly all carcinoma. In particular, peritoneal carcinomatosis is a common sign of advanced tumor stage of gastrointestinal or gynecological origin or of primary peritoneal malignancy like peritoneal mesothelioma or peritoneal carcinoma. It has been for long considered as a terminal disease with median survival about 6 months for colorectal carcinoma, 3 for gastric cancer, 2 for pancreatic cancer and 1.5 for carcinomatosis from unknown primary cancer and from 12 to 23 months for patient with stage 1V ovarian cancer.

Therapeutic approach was based on palliative systemic chemotherapy and surgery was mainly used in palliative intention except for ovarian cancer where it was part of the standard therapeutic regimen. Aim of surgery is to resect visible disease and chemotherapy aims at treating residual disease. Twenty years ago Sugarbaker introduced cytoreductive surgery (CRS) combined with hyperthermic intraperitoneal chemotherapy (HIPEC) as a new innovative therapeutic option for selected patients with peritoneal carcinomatosis. CRS procedure depends on the extent of the peritoneal disease and protocols of chemotherapy may include mytomycin, oxaliplatin, mitoxantrone, cisplatin alone or in combination. Moreover, HIPEC can be performed in open or closed abdomen technique and perfusion may vary from half to 2 hours. Although consensus about the ideal technique is not clear, CRS-HIPEC has been shown to improve survival of patients with peritoneal dissemination from colorectal cancer, gastric cancer, ovarian cancer, and diffuse malignant peritoneal mesothelioma. However, it is associated to relatively high mortality and morbidity resulting from surgery complications or from cytostatic agents toxicities including leucopenia, anemia, thrombopenia, heart, liver or renal toxicity.

Several studies have shown in rodents that RIT could be used efficiently as an adjuvant treatment after cytoreductive surgery in the treatment of peritoneal carcinomatosis. Numbers of intraperitoneal (i.p.) RIT studies using strong energy β- or α-emitting radionuclides are ongoing in animals. So far, five antibodies (anti-MUC1, CA-125, TAG-72 and gp38) have been conjugated to four β-emitting radionuclides for i.p. clinical application in patients suffering from ovarian cancer (Meredith R F, Buchsbaum D J, Alvarez R D, LoBuglio A F. Brief overview of preclinical and clinical studies in the development of intraperitoneal radioimmunotherapy for ovarian cancer. Clin Cancer Res. 2007; 13:5643s-5645s). Based on encouraging results (many phase I-II studies showed the efficiency of beta emitters like ¹³¹I, ¹⁷⁷Lu or ⁹⁰Y), a phaseIII randomized multicenter study has been undertaken and compared the efficiency of conventional chemotherapy with i.p. injection of Yttrium-90 labeled HMGF1 murine monoclonal antibody (anti-MUC1 mAb).

However it proved unsuccessful, as no improve in survival was observed after RIT, though peritoneal recurrence was significantly delayed. One explanation is that the irradiation dose delivered to tumors was not high enough. Indeed, non specific irradiation due to gamma-rays or to strong beta energy particles associated to conventional beta emitters is responsible for toxicities that make difficult repeating injections. In other words, the irradiation dose is at the same time too low for efficiently killing tumor cells and too high to be supported without damage by the organism.

Consequently, there is a need for an improved method of RIT suitable for peritoneal cancers.

### SUMMARY OF THE INVENTION

The present invention proposes in a first aspect a method of treating a subject having a cancer in a body cavity characterized in that it comprises the steps of:

administering in said body cavity of the subject radiolabeled binding molecules which bind to an antigen expressed by cancerous cells;
washing the body cavity to remove unbound radiolabeled molecules.

Advantageous but non limiting features are as follows:

said antigen expressed by cancerous cells is the carcinoembryonic antigen (CEA);
said binding molecules are monoclonal anti-CEA antibodies;
said antibodies are labelled with an Auger-emitter radionuclide;
said radionuclide is Iodine-125;
the activity of said administered radiolabeled binding molecules is above 1 GBq;
the activity of said administered radiolabeled binding molecules is above 100 GBq;
the peritoneal cavity is washed by a physiological saline solution;
the step of washing the peritoneal cavity is performed less than 24 hours, preferably one hour, after the step of administrating radiolabeled binding molecules;
the method further comprises a step of injecting in the blood of the subject additional radiolabeled binding molecules;
the step of injecting additional radiolabeled binding molecules is performed a few days, preferably between seven and eleven days, after the step of administering radiolabeled binding molecules in the peritoneal cavity of the subject;
the body cavity is the peritoneal cavity and the cancer is a peritoneal carcinomatosis;

In a second aspect, the invention proposes a body cavity perfusing system for carrying out the method according to the first aspect of the invention, the system comprising at least one inflow circuitry for injecting radiolabeled binding molecules and/or a washing solution, and at least one outflow circuitry for draining unbound radiolabeled molecules and washing solution.

### BRIEF DESCRIPTION OF THE DRAWINGS

The above, and other objects, features and advantages of this invention, will be apparent in the following detailed description of which is to be read in connection with the accompanying drawings wherein:

FIG. 1a represents bioluminescence imaging of tumors in the peritoneal cavity of a mouse with peritoneal carcinomatosis;

FIG. 1b-d represent Single-Photon Emission Computed Tomography (SPECT-CT) imaging of radioactivity distribution over time in the peritoneal cavity of a mouse treated with a method according to the invention;

FIG. 2 is a schematic view of a brief intraperitoneal RIT perfusing system;

FIG. 3 is a graph representing residual activity over time in the peritoneal cavity of mice treated with a method according to the invention;

FIG. 4 is a graph comparing survival rate over time of mice treated or not with a method according to the invention;

FIG. 5 is a graph comparing mean absorbed irradiation dose by organs of mice treated or not with a method according to the invention.

### DETAILED DESCRIPTION OF THE INVENTION

### Principe of the Invention

The method according to the invention aims at treating a subject having a cancer in a body cavity.

By body cavity is it meant any fluid-filled delimited space of an organism, generally lined with an epithelium. It designates in particular peritoneal cavity, but also thoracic cavity, cranial cavity, or subcavities like pleural cavity, pericardial cavity, etc. The invention will not be limited to any cavity.

The method according to the invention is a method of RIT which allows increasing the delivered irradiation dose while better protecting healthy tissues. To this end, after having administered in the body cavity of the subject radiolabeled binding molecules which bind to an antigen expressed by cancerous cells, the method according to the invention comprises a step of washing the body cavity to remove unbound radiolabeled molecules.

This step of washing the body cavity is advantageously performed less than 24 hours, preferably one hour, after the step of administrating radiolabeled binding molecules. It consists for example in an abundant flushing of the body cavity by a physiological saline solution (NaCl).

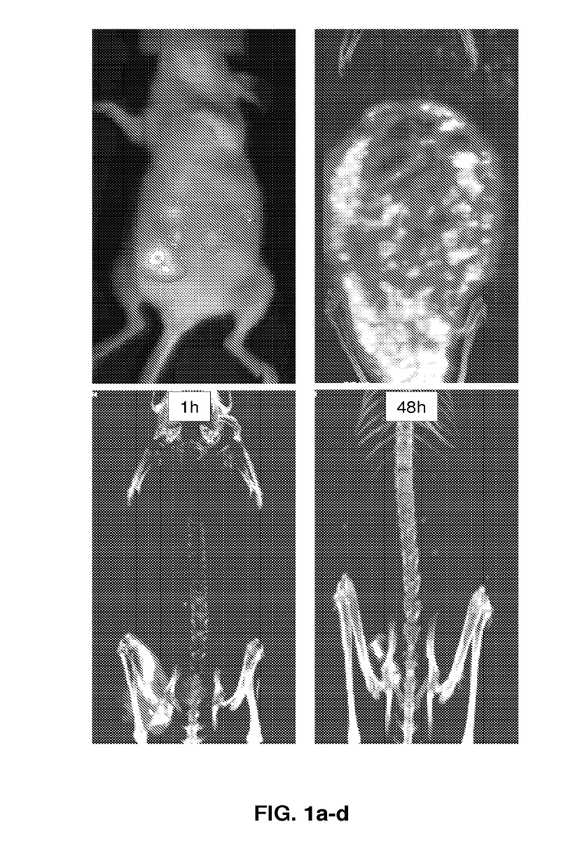

Indeed, as it can be seen in FIG. 1b (example of a mouse with tumors in the peritoneal cavity), after the step of administering radiolabeled binding molecules, radioactivity is distributed in the whole peritoneal cavity whereas tumors only occupy a few locations (especially in the down left corner here). Unbound radiolabeled molecules are consequently only damaging healthy tissues. By washing the cavity, unbound radiolabeled molecules are removed (see FIG. 1c), and so the major source of healthy tissues damaging is suppressed. The effect of the radioactivity is concentrated on tumors. As it can be seen, after 48 h the radioactivity exactly fits with the tumors (FIGS. 1a and 1d).

The method according to the invention, named Brief Radioimmunotherapy, and more particularly Brief Intraperitoneal Radioimmunotherapy (Bip-RIT) when the body cavity is the peritoneal cavity, allows consequently an increase of the administered irradiation dose while even lowering the effect on healthy tissues.

The following description will focus on peritoneal cavity as an example, it to be understood that a man skilled in the art will know how to adapt it to any cavity.

Monoclonal Antibodies (mAbs)

In RIT, the binding molecules which bind to an antigen expressed by cancerous cells are typically monoclonal (i.e. all identical) antibodies with specificity for a tumor-associated antigen, in particular the already mentioned mAbs (anti-MUC1, CA-125, TAG-72 and gp38).

However, different cancers often express different antigens. For peritoneal cancer, and in particular small volume peritoneal carcinomatosis, an interesting antigen is the Carcinoembryonic antigen (CEA). This is a glycoprotein involved in cell adhesion. It is normally produced during fetal development, but the production of CEA stops before birth. Therefore, it is not usually present in the blood of healthy adults, excepted in the blood of individuals with colorectal carcinoma, gastric carcinoma, pancreatic carcinoma, lung carcinoma or breast carcinoma. CEA measurement is mainly used as a tumor marker to identify recurrences after surgical resection, or localize cancer spread though dosage of biological fluids.

That is why the administered binding molecules are advantageously anti-CEA mAbs. In particular, the non-internalizing murine IgG1k 35A7 mAb, specific for the CEA Gold 2 epitope, is suitable.

### Radiolabeling

As already explained, mAbs are commonly labeled with strong energy β- or α-emitting radionuclides. A radionuclide thus undergoes radioactive decay and emits subatomic particles that constitute ionizing radiation. Among radionuclides, some decay by a physical phenomenon named “Auger effect” in which the transition of an electron in an atom filling in an inner-shell vacancy causes the emission of another electron (the “Auger electron”), presenting low energy and subcellular range. Such radionuclides are named Auger electron emitting radionuclides, or simply Auger emitters

The energy of Auger electrons is comprised between few eV and few keV and those having very low energy (<1 keV) behave like high linear energy transfer (LET) particles, namely alpha particles, with LET values ranging from 4 to 26 keV/μm. They are subsequently highly deleterious for biological materials because of highly localized energy deposits and this characteristic makes them good candidates to overcome radioresistance of solid tumors. Moreover, as explained their path length in biological matter is much shorter than for alpha particle since it is comprised between about 2 nm to 500 nm. The gain is that it produces minimal toxicity towards surrounding non-targeted cells. For these reasons they make possible repeated injections or combination with radiation synergistic chemotherapy.

Auger emitters could thus significantly delay growth of small intraperitoneal solid tumors, and consequently, the administered mAbs are advantageously labelled with an Auger emitter. ¹²³I, ¹²⁵I, ¹¹⁹Sb, etc. are example of Auger emitter. The preferred Auger emitter is ¹²⁵I. Therapeutic advantage of ¹²⁵I decays is that it produces soft X-rays together with cascades of Auger electrons (up to 21) at extremely low energies of 50 to 500 eV. Moreover, it decays to a stable nonradioactive ground-state product (Te-125).

So, Bip-RIT using ¹²⁵I-anti CEA mAb, was shown to produce a higher tumor-to-blood uptake ratio than conventional intravenous (i.v.) RIT. This is accompanied by a low toxicity for healthy tissues and by a significant increase in median survival. Therefore, the results suggest that Bip-RIT using ¹²⁵I-labeled anti CEA mAbs in combination with radiation synergistic drugs seems to be an interesting tool for the therapy of small peritoneal carcinomatosis as encountered after cytoreductive surgery.

Tests (that will be described below) involved ¹²⁵I from Perkin Elmer (Boston, Mass., USA) and mAbs radiolabeled at specific activity of 740 MBq/mg for RIT and biodistribution studies, using IODO-GEN method. Immunoreactivity of ¹²⁵I-mAbs against CEA was assessed in vitro by direct binding assays. The binding percentage was determined by measuring the antigen-bound radioactivity after 2 washes with PBS and was about 50-60%.

With regards to the activity of the administered dose of radionuclides, in the case of a human body it can exceed 1 GBq, and even 100 GBq. A dose of the order of 1 TBq may even be reached. Indeed, after washing, only a few percents of the administered dose may be kept within the body. Moreover, the method according to the invention is far less deleterious for healthy organs, and higher doses can be used. As an example, assuming that 93% of radionuclides are removed by washing, an initial activity of 300 GBq leads to a remaining activity of 21 GBq, i.e. 57 mCi.

### Supplementary Intravenous RIT

As it will also be further shown, the applicant surprisingly discovered that adding a supplementary step of injecting in the blood of the subject additional radiolabeled binding molecules (i.e. performing an auxiliary conventional intravenous RIT), brings even better results.

This “small” iv-RIT shall be performed a few days, preferably between seven and eleven days, after the step of administering radiolabeled binding molecules in the peritoneal cavity of the subject. Advantageously, same mAbs and radionuclides are used for both RIT, but the activity of the supplementary intravenous injected dose should be reduced (a few hundreds of MBq) with respect to the activity involved in a conventional iv-RIT.

### Therapeutical Experiments

Athymic nude mice (6-8 week/old females) were obtained from Charles River (Lyon, France) and were acclimated for 1 week before experimental use. They were housed at 22° C. and 55% humidity with a light/dark cycle of 12 h. Food and water were available ad libitum. The day before RIT, force-feeding with Iugol's solution was performed and stable iodine was added in drinking water and maintained during all the experimental protocol. Body weight was determined weekly and clinical examinations were carried out throughout the study. Hematologic toxicity was monitored during 70 d after onset of RIT, using scil vet ABC automate (SOIL animal care company, Altorf, France). All the animals experiments were performed in compliance with the French guidelines and standards of INSERM for experimental animal studies (Agreement no. B34-172-27).

For RIT experiments, mice were intraperitoneally grafted with 0.7×10⁶ A-431 cells (The vulvar squamous carcinoma cell line A-431 expressing the Epidermal Growth Factor Receptor (EGFR or HER1) and transfected with vectors encoding for the CEA and for luciferase genes was used) suspended in 0.3 ml DMEM medium.

Tumor growth was assessed 3 days after cell xenograft by bioluminescence imaging and allowed to segregated mice in homogeneous groups. Mice could be treated either by Bip-RIT, ip-RIT (without washing step), by intravenous RIT (iv-RIT) or by combination of Bip-RIT and iv-RIT (Bip+iv-RIT). The protocol used for Bip-RIT was the following: Mice were anaesthetized using intraperitoneal injection of a solution containing 100 mg/kg ketamine (Ketamine™ Panpharma; Panpharma, Fougere, France) and 1 mg/kg medetomidine (Dormitor™; Pfizer, Paris, France). Next, mice were intraperitoneally injected with either NaCl or ¹²⁵I-mAbs in a final volume of 5 mL. The non-internalizing murine IgG1 k 35A7 mAb, specific for the CEA Gold 2 epitope, was used to target CEA. The irrelevant PX antibody was used for control experiments. PX is an IgG1 mAb that has been purified from the mouse myeloma MOPC 21. The 35A7 and PX mAbs were obtained from mouse hybridoma ascites fluids by ammonium sulfate precipitation followed by ion exchange chromatography on DE52 cellulose (Whatman, Balston, United Kingdom).

One hour after injection, drug was removed and the cavity flushed with 25 mL NaCl for 15 min. Once wash of the peritoneal cavity was achieved, catheters were removed and mice were weighted. Mice were then awaked by i.p. injection of Atipamezole™ (Antisedan 2.5 mg/kg body weight, Pfizer, Paris, France). Ip-RIT consisted of standard intraperitoneal injections with volume of 5 mL without wash of the peritoneal cavity and iv-RIT was conventionally done.

Thus, four days after graft, one group of mice was injected with 5 mL of NaCl according to Bip-RIT methodology (namely Bip-NaCl-RIT). Another control group received intraperitoneal injection of 5 mL of NaCl according to standard ip-RIT methodology (ip-NaCl-RIT) One group was treated with Bip-RIT using ¹²⁵I-35A7 mAb (Bip-¹²⁵I-35A7-RIT) alone while two others received Bip-RIT ¹²⁵I-35A7 and additional i.v. injection at day 7 (Bip+ivd7-¹²⁵I-35A7-RIT), or at day 11 (Bip+ivd11-¹²⁵I-35A7-RIT). In order to assess non-specific efficiency of ¹²⁵I-mAbs, another group was treated with Bip-RIT using ¹²⁵I-PX mAb followed by i.v. injection of ¹²⁵I-PX mAb at day 7 (Bip+ivd7-¹²⁵I-PX-RIT). The last group received two i.p. injections of ¹²⁵I-35A7 mAb at day 4 and 7 (Ip-¹²⁵I-35A7-RIT). In summary, 8, 7, 10, 7, 9, 7 and 15 mice were included in the groups, respectively.

Tumor growth was followed weekly by bioluminescence imaging. Mice were sacrificed when the bioluminescence signal reached a value of 4.5×10⁷ photons/s corresponding to total tumor weight about 0.2-0.3 g.

In vivo bioluminescence imaging was performed following i.p. injection of luciferin (0.1 mg luciferin/g). Whole-body SPECT/CT images were acquired at various times following Bip-¹²⁵I-35A7-RIT (Oh, 1 h, 24 h, 48 h and 72 h) with a two-headed multiplexing multi-pinhole NanoSPECT (Bioscan Inc., Washington D.C.). The pinholes aperture was 1 mm. Energy window was centered at 28 keV with ±20% width, acquisition times were defined to obtain 30 000 counts for each projection with 24 projections. Images and maximum intensity projections (MIPs) were reconstructed using the dedicated software Invivoscope® (Bioscan, Inc., Washington, USA) and Mediso InterViewXP® (Mediso, Budapest Hungary). Concurrent microCT whole-body images were performed for anatomic coregistration with SPECT data. Reconstructed data from SPECT and CT were visualized and co-registered using Invivoscope®. As already mentioned, FIGS. 1a-d represent examples of acquired images.

On day 1, 48 athymic nude mice were intraperitoneally grafted with 0.7×10⁶ A-431 cells suspended in 0.3 ml DMEM medium. Mice were divided in two groups in order to compare biodistributions of ¹²⁵I-35A7 mAb following either Bip-RIT or iv-RIT. Then first group of mice was treated by Bip-¹²⁵I-35A7-RIT according to the previously described methodology but the injected solution was made of 5.5 MBq (740 MBq/mg) of ¹²⁵I-35A7 mAb completed with 243 μg of unlabeled 35A7 mAb diluted in 5 mL to mimic therapeutic activity of 185 MBq (740 MBq/mg). This group was called Bip-¹²⁵I-35A7-Biodis.

The second group was intravenously injected with a solution containing 185 MBq (740 MBq/mg) of ¹²⁵I-35A7 mAb completed with 50 μg of unlabeled 35A7 mAb diluted in 300 μL of saline solution (iv-¹²⁵I-35A7-Biodis) to mimic therapeutic activity of 37 MBq (740 MBq/mg).

For the two groups, mice were sacrificed at 1, 24, 48, 72, 96, 144 and 168 h after Bip- or iv-Biodis. At each time point, animals were anaesthetized, image acquisition by bioluminescence was performed and then they were euthanized, bled and dissected. Blood and other healthy organs were weighed. However, as described in Santoro et al. (20), for tumor nodules, size was first determined in order to calculate tumor volume and thereby tumor weight considering density of 1.05 g/cm³. Uptake of radioactivity during biodistribution experiments (i.e., UOR_{Biodis}) was next measured for tumor nodules and for all the organs with a gamma-well counter. The percentage of injected activity per gram of tissue (% IA/g), corrected for the radioactive decay, was calculated for iv-¹²⁵I-35A7-Biodis. For Bip-¹²⁵I-35A7-Biodis, results were expressed in term of percentage of remaining activity per gram of tissue (% RA/g), immediately after wash out with saline solution (i.e. one hour post injection).

Since accurate direct measurement of weight of i.p. tumor could not be performed in RIT experiments because it requires mice sacrifice and also because of the high activities, then it was assessed from weekly bioluminescence signal. For this purpose, it was performed biodistribution experiments for determining the calibration curve between the bioluminescence signal of tumors and their size. Typically, prior to sacrifice, tumors were imaged by bioluminescence and the corresponding signal (photon/s) was correlated with calculated tumor weight (g) determined itself from direct measurement of tumor nodules size.

The uptake of radioactivity per tissue (expressed in Becquerel) in RIT experiments (UOR_{RIT}) was extrapolated from the uptake per tissue (UOR_{Biodis}) measured during biodistribution experiments. Since activities used in RIT experiments were 33 times higher than those used in biodistribution analysis for the same amount of injected mAbs (250 μg), all the UOR_{Biodis} values were multiplied by 33 to mimic the therapeutic conditions. The weight of healthy tissues was considered not to change all along the study period and did not differ between RIT and biodistribution experimental conditions. It was checked that this assumption was also true for tumor nodules during the first week after injection. Therefore, the 33-fold factor's rule was enough to determine the UOR_{RIT} from UOR_{Biodis}.

The total cumulative decays per tissue were calculated by measuring the area under the UOR_{RIT} curves. Following the MIRD formalism, resulting values were multiplied by the S factor. This parameter was calculated by assuming that all the energy delivered at each decay was locally absorbed and it was checked that the contribution of X and y-rays could be neglected. A global energy of 19.483 keV/decay was then considered for calculating the irradiation doses.

A linear mixed regression model (LMRM), containing both fixed and random effects, was used to determine the relationship between tumor growth (assessed by bioluminescence imaging) and number of days post-graft. The fixed part of the model included variables corresponding to the number of post-graft days and the different mAbs. Interaction terms were built into the model; random intercepts and random slopes were included to take into account time. The coefficients of the model were estimated by maximum likelihood and considered significant at the 0.05 level.

Survival rates were estimated from the date of the xenograft until the date of the event of interest (i.e., a bioluminescence value of 4.5×10⁷ photons/s) using the Kaplan-Meier method. Median survival was presented and survival curves compared using the Log-rank test. Statistical analysis was performed using the STATA 10.0 software.

### Experimental Results

Tests demonstrate the technical feasibility of brief intraperitoneal RIT (Bip-RIT) in mice using high activity of ¹²⁵I-mAbs. Biodistribution study shows that 4 days after graft, i.e. prior to therapy, the mean diameter of tumor nodules was about 1.5-2 mm and that about 5-6 nodules were detected per mouse. This corresponds to mean tumor weight of 1.2±0.9×10⁻² g. In Bip_NaCl treated group, tumor grew exponentially and all mice were sacrificed before day 40. Similar growth rate was obtained for mice treated by Bip-¹²⁵I-PX-RIT. The highest delay in tumor growth was obtained for Bip+ivd7-¹²⁵I-35A7-RIT group and intermediary tumor growth kinetic was obtained for Bip+ivd11-¹²⁵I-35A7-RIT and Ip-¹²⁵I-35A7-RIT groups.

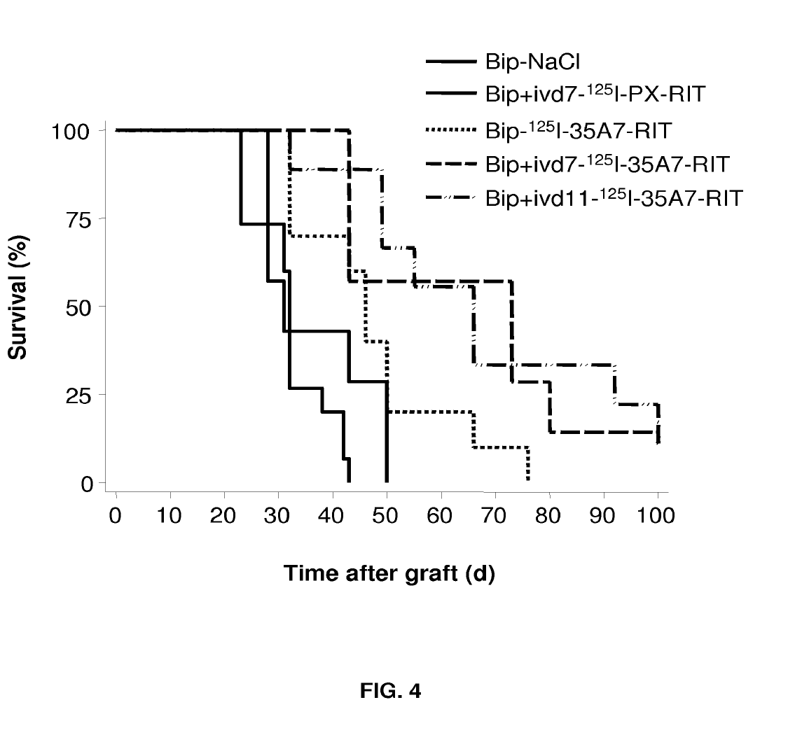

We observed that wash of the peritoneal cavity with NaCl slowed down by itself, tumor growth of the Bip-NaCl-RIT group compared to ip-NaCl-RIT group. This was confirmed by a lower median survival of mice treated by ip-NaCl-RIT (FIG. 4).

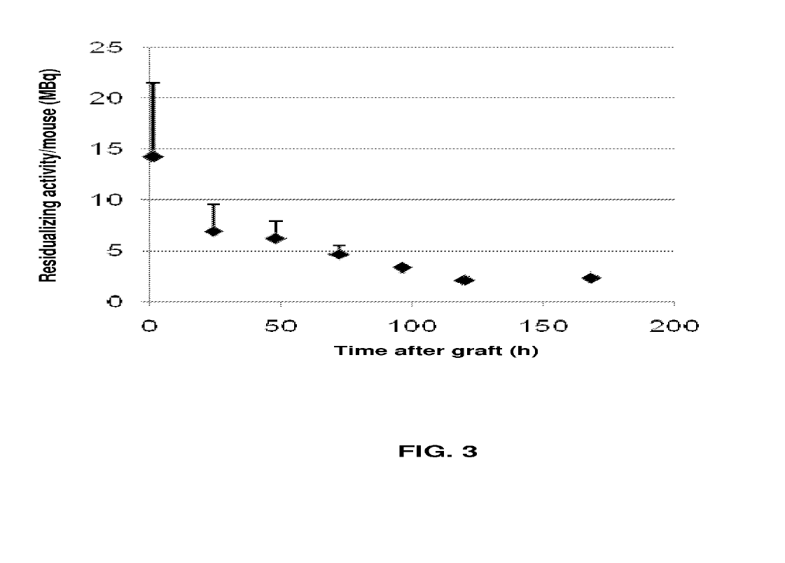

Residual activity per mice was about 14.2±7.3 MBq immediately after wash with saline solution and dropped to 2.1±0.7 MBq at day 5 after injection (FIG. 3). These results indicate that about 7.6% of the injected activity was effectively kept within mice. No weight loss was observed after Bip-RIT. These results suggest that the latter methodology is well tolerated by mice. However mild and transient hematological toxicity was observed in all treated mice. All the values are expressed relatively to the control at the considered time. For Bip-¹²⁵I-35A7-RIT treated mice, nadir for lymphocytes and monocytes was reached between days 7 and 10 (around −50%) after graft. Decrease in platelets occurred slightly later (day 15, −30%), while no obvious decrease was observed for granulocytes and red blood cells. Most of the values returned to normal values around day 39. When mice received additional intravenous injections of ¹²⁵I-mAbs at day 7 (Bip+ivd7-¹²⁵I-35A7-RIT, Bip+ivd7-¹²⁵I-PX-RIT), decrease was found to be more pronounced and prolonged for lymphocytes and monocytes with nadir occurring between day 10 and 22 after graft (about −75%). Decrease in granulocytes and platelets was also observed with nadir at day 22 (−30%-−50%) and at day 15 (−50%), respectively. Finally most of the values returned to standard values or started increasing on day 39. When additional i.v. injection was done at day 11 after graft, hematological toxicity was in the same range but was more maintained with time and values were not still returned to control on day 39. For later times, ratio could not be calculated since most of the Bip-NaCl treated mice were sacrificed because of tumor growth.

It must be noted that no difference was observed between ¹²⁵I-PX and of ¹²⁵I-35A7 mAbs suggesting that medullar toxicity was mainly due to non specific irradiation, including soft X-rays or the most energetic electrons emitted by ¹²⁵I. In addition, it was shown that ip-¹²⁵I-35A7-RIT consisting of two injections of 37 MBq of ¹²⁵I-35A7 at days 4 and 7 produced lower toxicity than Bip+ivd7-¹²⁵I-35A7-RIT while lower activities were finally present in mice in the latter case. This result suggests that the high activity of 185 MBq maintained for one hour is mainly responsible for hematological toxicity.

As already mentioned, SPECT-CT imaging showed a good fitting bioluminescence signal and that radioactivity was homogeneously distributed in the peritoneal cavity after injection (FIG. 1b). Wash of the peritoneal cavity was accompanied by a concentration of the latter radioactivity at the tumor nodules level and was observed at least until day 3 after injection (FIG. 1d). Uptake of radioactivity by tumor nodule was as determined during biodistribution study and extrapolated to RIT experiments, as described below. It was thus calculated that the activity contained in tumor nodules was about 0.1 MBq/mouse immediately after wash was completed.

Biodistribution study confirms a strong uptake of ¹²⁵I-35A7 mAb by tumor nodules. The percentage of residual activity/g of tumor (% RA/g) immediately after wash out ranged between 72.1±30.2% at 1 h and 20.5±4.8% at 168 h. The latter values were much higher than peak values of 27.8±7.2% of the injected activity/g of tumor (% IA/g) determined after a single i.v. injection of 37 MBq (740 MBq/mg) characterizing iv-¹²⁵I-35A7-RIT. In addition, uptake of radioactivity by healthy organs was shown to be higher after i.v. injection. Peak value of % RA/g of blood was observed at 1 h and was shown to be 12.2±3.2% while it was about 28.1+2.4% after iv_{—}¹²⁵I-35A7-RIT. These results suggest that Bip-¹²⁵I-35A7-RIT methodology both improved tumor targeting and partially protected healthy tissues compared to i.v. injection.

Mice were sacrificed when bioluminescence signal reached 4.5×10⁷ photons/s corresponding to mean tumor weight of about 0.2-0.3 g. Median survival (MS) was about 31 d in the Bip-NaCl-treated group and was increased up to 49 days in the group treated with Bip-¹²⁵I-35A7-RIT. This value was significantly improved when additional intravenous injections were added at day 7 (Bip+ivd7-¹²⁵I-35A7-RIT) and 11 (Bip+ivd11⁻¹²⁵I-35A7-RIT) with MS reaching 73 d and 66 d days, respectively (FIG. 4). No statistically significant difference (MS=31 d) was observed when mice were treated with Bip+ivd7-¹²⁵I-PX-RIT suggesting thereby the lack of toxicity/efficiency of ¹²⁵I when unbound to the cells. Median survival was about 49 d after two standard i.p. injections with ¹²⁵I-anti CEA mAb (ip-¹²⁵I-35A7-RIT) and corresponding MS in mice receiving two standard injections of NaCl was about 23 d, which confirms that Bip-¹²⁵I-anti CEA mAb-RIT improves survival of mice.

From biodistribution data, uptake of radioactivity (UOR) of ¹²⁵I-35A7 mAb by safe organs and tumor nodules has been expressed as a function of time. For most of the tissues, UOR was shown to be maximal at 1 h i.e. immediately after wash associated to Bip-RIT, and was next shown to decrease. UOR was higher after iv-RIT than after Bip-RIT for skin (3.3×10⁵ versus 6.5×10⁵ Bq), kidneys (2.8×10⁵ versus 9.6×10⁵ Bq), large intestine (2.3×10⁵ versus 4.16×10⁵ Bq) and small intestine (3.9×10⁵ versus 8×10⁵ Bq), tumor (4.4×10⁴ versus 2.0×10⁵ Bq), heart, bones and spleen. In particular, it was much lower for blood (1.0×10⁶ versus 7.4×10⁶ Bq) and liver (5.1×10⁵ versus 4.3×10⁶ Bq) and carcass (8.9×10⁶ versus 1.9×10⁷ Bq). It was only higher after Bip-RIT for stomach (1.1×10⁶ Bq versus 2.3×10⁵ Bq). Peak of tumor uptake (1.2×10⁵ Bq was shown to be reached immediately after injection Bip-RIT and slowly decreased down to 5.0×10⁴ Bq at 168 h. By contrast peak values (2.0×10⁵-2.1×10⁵ Bq) were obtained between 24 h and 48 h after iv-RIT. Corresponding value was 1.2×10⁵ Bq at 168 h.

Cumulated uptake of radioactivity (CUOR) was next calculated by measuring area under the obtained curves.

According to MIRD formalism, the mean absorbed irradiation dose per organ was calculated by multiplying the CUOR by S-value corresponding to ¹²⁵I.

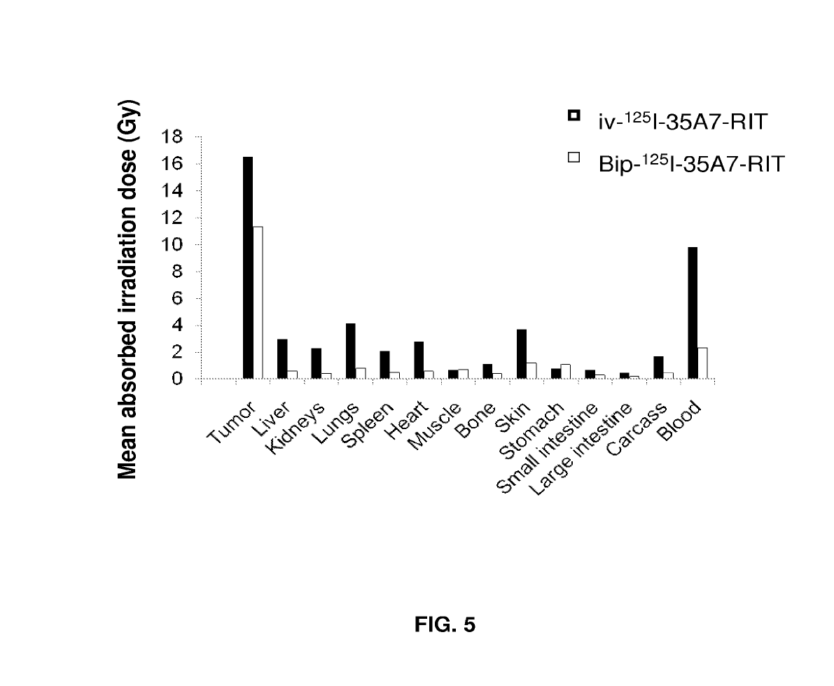

Regarding dosimetry (see FIG. 5), mean absorbed irradiation dose by tumor was shown to be 11.6 Gy for Bip-RIT ¹²⁵I-35A7 while a value of 16.7 Gy was calculated after iv-RIT. However, dose delivered to healthy organs was shown to be much lower with dose to the blood about 1.9 Gy while iv-RIT delivered about 9.8 Gy. Irradiation dose to the other organs did not exceed 1 Gy.

### Simulation of Human Body

Considering human body size and X-rays energy, ¹²⁵I toxicity is not expected to be penalizing for clinical application. Indeed, another test used an anthropomorphic phantom where peritoneal cavity was replaced by a cubic volume containing 4 L (3.7 GBq) of ¹²⁵I-mAb, and loaded with lithium fluoride thermoluminescent dosimeter.

It was determined a dose rate about 300 μSv.h⁻¹ at the sacrum level. Considering an incubation period of one hour, about 300 μSv would be delivered by external irradiation to hematopoietic areas. Therefore, hematological toxicity is expected to be much lower than in mice.

It should be noted that as the weight of a person is about 2000 times the weight of a mouse (30 g), the afore-mentioned activity of 185 MBq would be proportionally equivalent for an average human body to an activity of the order of 370 GBq.

Considering activity contained in organs, uptake of radioactivity by blood and healthy tissues was low and peak uptake values (Bq/g tissue) were obtained for the tumor nodules. SPECT-CT imaging corroborated these results since images showed that radioactivity was concentrated at the tumor level. This confirmed the generally described advantage of i.p. over i.v. drugs administration in terms of concentration and tolerance. Therefore, although reducing potential reservoir of ¹²⁵I-mAb that may constitute blood after iv-RIT, Bip-RIT procedure including wash out of the peritoneal cavity eliminates, by the same time, undesirable radioactivity.

Although irradiation dose delivered to the tumor was lower after Bip-RIT than after iv-RIT, tumor-to-blood irradiation dose was about 5 and 1.7 for Bip-RIT and iv-RIT, respectively. Irradiation dose to healthy organs by Bip-RIT was generally very low since it did not exceed 1 Gy. By contrast, 4.2 Gy or 3 Gy, for example, could be achieved with iv-RIT for lung and liver, respectively, which are after blood, the most exposed organs. These results indicated that Bip-RIT with ¹²⁵I-mAbs protect healthy tissues while delivering significant irradiation dose to the tumor. It was thus observed that survival of treated mice was significantly improved after Bip-RIT alone. However, this result was significantly better when additional iv-¹²⁵I-anti-CEA mAb-RIT was done. MS was thus increased from 31 d to 73 d. The latter values might be compared to increase in MS from 20 d to 59 d determined in our previous study after two i.v. injections of 37 MBq of ¹²⁵I-anti CEA mAbs for treating similar tumors. If the above described non specific hematological toxicity was effectively due to the initial one-hour incubation time of the peritoneal cavity with ¹²⁵I-mAbs, one can consider that repeated injections of ¹²⁵I-mAbs could be planed or that their combination with chemotherapy may be possible, without significant toxicities. When comparing Bip+ivd7-RIT or Bip+ivd11-RIT with ip-RIT using two injections of ¹²⁵I-anti CEA mAb, it was shown that MS was improved from 23 to 49 d with low associated hematological toxicity. This increase in survival was similar to what was observed after Bip+ivd7-RIT (from 32 to 73 d). However, it must be kept in mind that uptake of radioactivity by healthy tissues during Bip-RIT was very low and probably much lower than during ip-RIT. In addition, combining Bip- and iv-RIT takes advantage of the better tumor uptake obtained with i.p. route while delayed i.v. injection may allow to reach pockets of target cells that are not targeted by i.p administration.

Completeness of resection as well as the tumor load were shown to be the most important factors predictive of long-term survival after CRS-HIPEC. In the present study, targeted tumor nodules were of about 1.2±0.9×10⁻² g. It was demonstrated with β-emitters that RIT must be dedicated to small solid tumors. This is even more pronounced when low energy electrons emitters (especially Auger emitters) are used. However, the efficiency of ¹²⁵I in delaying tumor growth is rather unexpected if one considers that path length of electrons emitted by ¹²⁵I ranges from nm to about 20 μm compared to tumor size of several mm diameter. Our results may be compared to those obtained by Aarts et al. in rats bearing CC-531 colon carcinoma tumor xenograft of few mm and i.p. injected with a single activity of 74 MBq of ¹⁷⁷Lu-labeled MG1 mAb. They showed that MS was strongly increased from 57 to 97 d when RIT was combined to CRS.

### CONCLUSION

The study proves that Bip+iv-¹²⁵I-anti CEA mAb-RIT are an efficient tool in the therapy of small peritoneal carcinomatosis as those encountered after cytoreductive surgery. It confirmed the efficiency of ¹²⁵I-anti CEA mAb in killing tumor cells. Bip+iv-RIT takes advantage of a strong tumor-to-healthy tissues and of low toxicity of ¹²⁵I decay for non targeted tissues. It makes possible to increase injected activities or to repeat injections or to combine RIT with radiation synergistic agents such as taxol, or drugs targeting microenvironment.

### Body Cavity Perfusing System

According to a second aspect, a body cavity perfusing system for carrying out the method according to the first aspect of the invention is providing, the system comprising at least one inflow circuitry 11 for injecting radiolabeled binding molecules and/or a washing solution, and at least one outflow circuitry 21a, 21b for draining unbound radiolabeled molecules and washing solution.

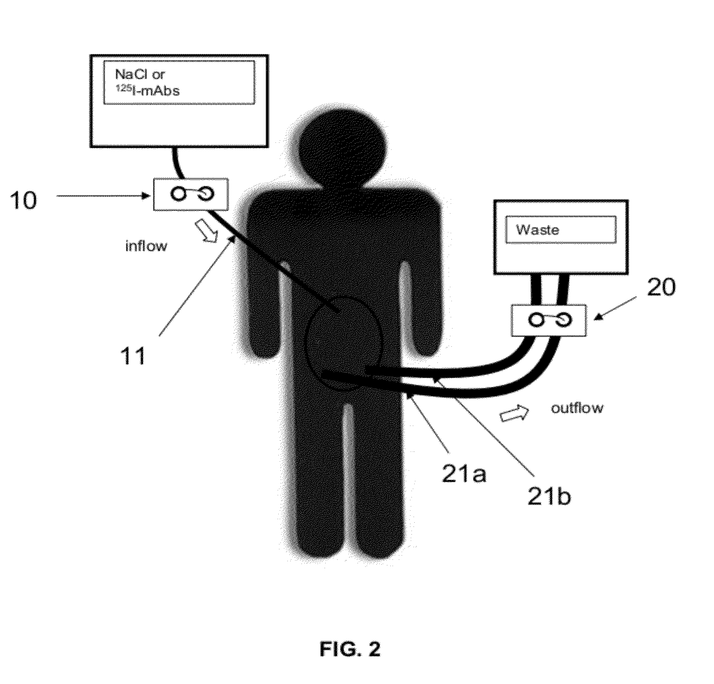

An example of such a perfusing system is represented by the FIG. 2, the body cavity being the peritoneal cavity (in other words, this system is a peritoneal perfusing system). Typically, an inflow needle is placed in the upper part of the abdominal cavity and two multiperforated catheters were inserted laterally through the abdominal wall are used at outflows. The system is not limited to a specific number of inflows and/or outflows.

Perfusion may be done using one or more pumps 10, 20, preferably peristaltic pumps.

1. A method of treating a subject having a cancer in a body cavity characterized in that it comprises the steps of:
administering in said body cavity of the subject radiolabeled binding molecules which bind to an antigen expressed by cancerous cells;
washing the body cavity to remove unbound radiolabeled molecules.

2. A method according to claim 1, wherein said antigen expressed by cancerous cells is the carcinoembryonic antigen (CEA).

3. A method according to claim 2, wherein said binding molecules are monoclonal anti-CEA antibodies.

4. A method according to claim 1, wherein said antibodies are labelled with an Auger-emitter radionuclide.

5. A method according to claim 4, wherein said radionuclide is Iodine-125.

6. A method according to one of claim 4 or 5, wherein the activity of said administered radiolabeled binding molecules is above 1 GBq.

7. A method according to claim 6, wherein the activity of said administered radiolabeled binding molecules is above 100 GBq.

8. A method according to claim 1, wherein the peritoneal cavity is washed by a physiological saline solution.

9. A method according to claim 1, wherein the step of washing the peritoneal cavity is performed less than 24 hours, preferably one hour, after the step of administrating radiolabeled binding molecules.

10. A method according to claim 1, further comprising a step of injecting in the blood of the subject additional radiolabeled binding molecules.

11. A method according to claim 10, wherein the step of injecting additional radiolabeled binding molecules is performed a few days, preferably between seven and eleven days, after the step of administering radiolabeled binding molecules in the peritoneal cavity of the subject.

12. A method according to claim 1, wherein the body cavity is the peritoneal cavity and the cancer is a peritoneal carcinomatosis.

13. A body cavity perfusing system for carrying out the method according to claim 1, the system comprising at least one inflow circuitry (11) for injecting radiolabeled binding molecules and/or a washing solution, and at least one outflow circuitry (21a, 21b) for draining unbound radiolabeled molecules and washing solution.
